(19) United States
(12) Patent Application Publication
(10) Pub. No.: US 2015/0175462 A1
(43) Pub. Date: Jun. 25, 2015

(54) Method for Promoting Production of Biogas Using Pancreatin in an Anaerobic Digestion Process

(71) Applicant: Korea Advanced Institute of Science and Technology, Daejeon (KR)

(72) Inventors: Woong Kim, Daejeon (KR); Young-Back Kim, do (KR); Jae-Hyun Yoon, Gyeongsangbuk-do (KR); Hyeong-Jun Roh, Gyeongsangbuk-do (KR); Han-Seok Noh, Gyeongsangbuk-do (KR)

(73) Assignee: Korea Advanced Institute of Science and Technology, Daejeon (KR)

(21) Appl. No.: 14/412,993

(22) PCT Filed: Jul. 3, 2013
(86) PCT No.: PCT/KR2013/005883

§ 371 Date: Jan. 5, 2015

(30) Foreign Application Priority Data
Jul. 4, 2012 (KR) 10-2012-0072675

(51) Int. Cl. C02F 11/04; C12N 9/94

(52) U.S. Cl. CPC C02F 11/04; C12N 9/94

(57) ABSTRACT

Disclosed is a method for promoting production of biogas using pancreatin in an anaerobic digestion process. In addition, disclosed are a composition for improving hydrolysis efficiency or promoting production of biogas, which includes pancreatin as an active ingredient, and a method for promoting (or increasing) production of biogas from organic manure of livestock using the same. The composition for improving hydrolysis efficiency or for promoting production of biogas in an anaerobic digestion process for treatment of organic waste, includes pancreatin as an active ingredient. The organic waste is livestock manure, and the composition has optimum activity at pH 7.0 to pH 8.0. The composition further includes a microorganism with excellent degradability of degradation resistant organic compounds.

### RELATED APPLICATIONS

This application claims priority to Korean Patent Application No. 10-2012-0072675, filed on Jul. 4, 2012 in the Korean Intellectual Property Office, the entire disclosure of which is incorporated herein by reference.

### BACKGROUND OF THE INVENTION

1. Field of the Invention

The present invention relates to a method for promoting production of biogas using pancreatin in an anaerobic digestion process, and more particularly, to a composition for improving hydrolysis efficiency or promoting production of biogas, which includes pancreatin as an active ingredient, and a method for promoting (or increasing) production of biogas from organic manure of livestock using the same.

2. Description of the Related Art

Organic waste may include livestock manure, food waste, residues of plants such as greens or vegetables, agricultural by-products, by-products of food and paper manufacturing facilities or the like. Although these wastes are sometimes recycled in the forms of feedstuff or fertilizer by a variety of treatment processes, other methods such as landfill or sea disposal may cause environmental pollution. In particular, according to “London Dumping Convention protocol 1996,” dumping of organic waste at sea will be completely forbidden in 2012, therefore, a biogas plant industry, which is an alternative proposal for treatment of organic waste, has become increasingly important as a national undertaking.

With regard to livestock manure in the country, about 80% thereof is recycled into resources such as compost or liquid fertilizer. However, if dumping of livestock manure at sea is prohibited since 2012, it is inevitable that all livestock manure will be treated on land. Livestock manure generally includes a high concentration of organic compounds and may contaminate rivers and lakes, if it is discharged without additional treatment processing. Specifically, nitrogen and phosphorous contained in great quantities therein may induce eutrophication in a water system, which in turn, may cause drinking water and stream water such as agricultural water to be useless.

Meanwhile, biogas refers to a gas generated during degradation of organic matter (‘organics’), for example, food garbage, waste, livestock manure, etc., by an anaerobic microorganism in an oxygen-free status (anaerobic state) and mostly includes methane gas and carbon dioxide as main components.

Biogas generating technologies will be described in detail. In recent years, techniques for energy resource utilization using organic wastes have been continuously developed. For instance, a method for production of biogas through anaerobic digestion of organic waste was disclosed, and the produced biogas may be used by converting it to electrical or thermal energy. In addition, a by-product obtained from organic waste can be used such as fertilizer. Therefore, the above method has high utility and thus needs further development.

In particular, studies on the production of methane gas from food garbage or livestock manure and recycling thereof, i.e., anaerobic digestion, as part of a resource recovery process from organic waste, have been actively progressed. For instance, Korean Patent Application Laid-Open No 10-2011-0019079 discloses a method for promoting production of biogas in continuous thermophilic temperature single-phase anaerobic fermentation system using microbial pre-treatment, and Korean Patent Application Laid-Open No 10-2011-0129210 discloses an apparatus for treating organic waste having improved production yield of methane and a method thereof.

Anaerobic digestion typically refers to a process in that biologically degradable organics are degraded into carbon dioxide and methane in an oxygen-free state (‘anaerobic state’), however, practically includes a complicated reaction which is involved with a substrate-dependent reaction route and various microorganisms. Anaerobic degradation of converting solid organics to methane may have hydrolysis, acidogenesis and methanogenesis stages, separately.

According to theory, about 90% of bio-degradable organics in the organic waste may be converted to methane. Further, since sludge generated after stabilization of organic waste and anaerobic digestion thereof does not environmentally and adversely affect soil and water, and in addition, can be used as a soil conditioner and fertilizer, energy consumption may be further reduced. Moreover, methane is a main component of biogas and may be converted to a renewable energy in a high versatile form, such as heat and electrical energy, and also be used as an automotive fuel.

Consequently, anaerobic digestion may reduce a potential risk to global climate change by the following two methods. First, methane has about a 21-fold greater potential contribution to global warming than carbon dioxide, and accounts for about 15% of overall greenhouse gas emission in Korea. In this regard, the emission of methane in a natural state may be reduced by capturing biogas through anaerobic digestion. Second, if the biogas produced through anaerobic digestion can replace fossil fuels, it is possible to avoid generation of carbon dioxide from chemical fuel, thus decreasing a causal material of global warming.

However, conventional anaerobic digestion processes have several problems or limitations such as low growth of microorganisms, a bad smell of treated effluent, a great amount of buffer required for pH control, etc., which are difficult to be overcome by existing technological skills. Therefore, such processes as described above have drawn little attention until now, as compared to aerobic treatment processes.

Accordingly, the present inventors have studied improvement in efficiency of an anaerobic digestion process and found through experiments that hydrolysis of organics may be very effectively conducted, which in turns considerably shortens a waste treatment process, and promotes generation of biogas at the same time, if pancreatin is used for treatment of livestock manure. As a result, the present invention has been completed successfully.

### SUMMARY OF THE INVENTION

Accordingly, an object of the present invention is to provide a composition for improving hydrolysis efficiency or promoting production of biogas, in an anaerobic digestion process for treatment of organic waste.

Another object of the present invention is to provide a method for treatment of organic waste using the above composition, to efficiently conduct hydrolysis of the organic waste while promoting production of biogas.

In order to accomplish the above objects of the present invention, there is provided a composition for improving hydrolysis efficiency and promoting production of biogas in an anaerobic digestion process for treatment of organic waste, which includes pancreatin as an active ingredient.

According to one embodiment of the present invention, the organic waste may be livestock manure.

According to another embodiment of the present invention, the composition may have optimum activity at pH 7.0 to pH 8.0.

According to another embodiment of the present invention, the composition may further include a microorganism having excellent degradability of typically degradation resistant organic compounds.

In addition, the present invention provides a method for treatment of organic waste by an anaerobic digestion process, which includes adding a seed mixture as well as the composition containing pancreatin as an active ingredient to the organic waste and then conducting a reaction thereof.

According to one embodiment of the present invention, the organic waste may be livestock manure.

According to another embodiment of the present invention, the composition containing pancreatin as an active ingredient may include pancreatin at a concentration of 1 to 100 μg/ml.

According to another embodiment of the present invention, the composition containing pancreatin as an active ingredient may have effects of improving hydrolysis efficiency or promoting production of biogas.

According to another embodiment of the present invention, the composition containing pancreatin as an active ingredient may have optimum activity at pH 7.0 to pH 8.0.

According to another embodiment of the present invention, the composition containing pancreatin as an active ingredient may further include a microorganism with excellent degradability of degradation resistant organic compounds.

Since the inventive composition containing pancreatin as an active ingredient has effects of improving hydrolysis efficiency and promoting production of biogas in an anaerobic digestion process for treatment of organic waste, the composition of the present invention containing pancreatin as described above may be useful for treatment of organic waste using the anaerobic digestion process. Specifically, pancreatin used herein as an active ingredient exhibits optimum activity at pH 7.0 to pH 8.0, therefore, when pancreatin is used in a single-phase system wherein an organic compound is placed in a single digestion bath to conduct anaerobic digestion, the above active ingredient has a pH range substantially identical to an optimum pH range for methane fermentation, that is, pH 7.0 to pH 8.0 As a result, the present invention may be more effectively used in the single-phase system.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic view simply illustrating an anaerobic digestion process;

FIG. 2 is pictures showing experimental equipment used in an experiment of the present invention;

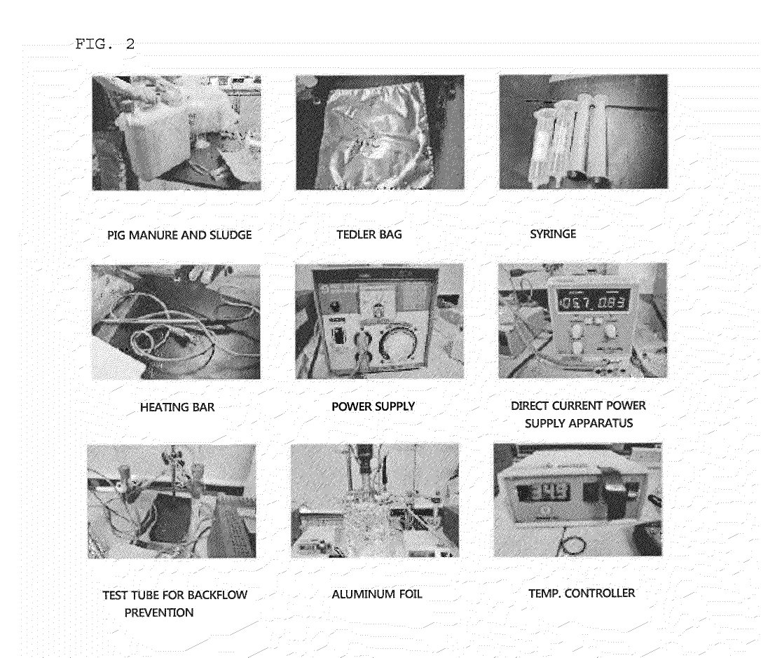

FIG. 3 is a schematic view simply illustrating an experimental device used in an experiment of the present invention;

FIG. 4 is pictures showing addition of pancreatin to a reactor containing an experimental group in an experiment of the present invention;

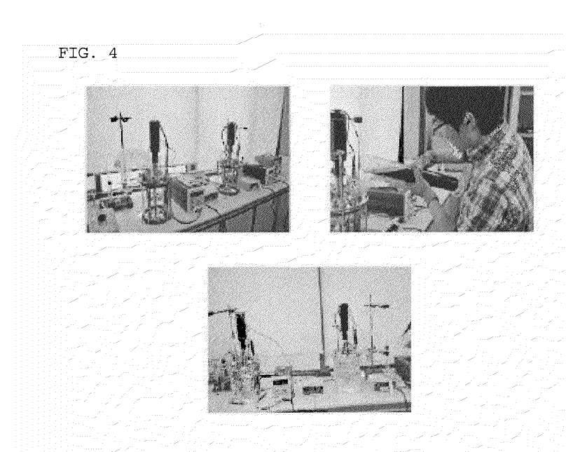

FIG. 5 is pictures showing procedures for organic acid measurement in an experiment of the present invention; and

FIG. 6 is pictures showing procedures for COD measurement in an experiment of the present invention.

### DETAILED DESCRIPTION OF THE INVENTION

The present invention relates to a novel use of pancreatin capable of improving hydrolysis efficiency in an anaerobic digestion process for treatment of organic waste, and more particularly, to provision of a composition for improving hydrolysis efficiency and promoting production of biogas in an anaerobic digestion process for treatment of organic waste, which includes pancreatin as an active ingredient.

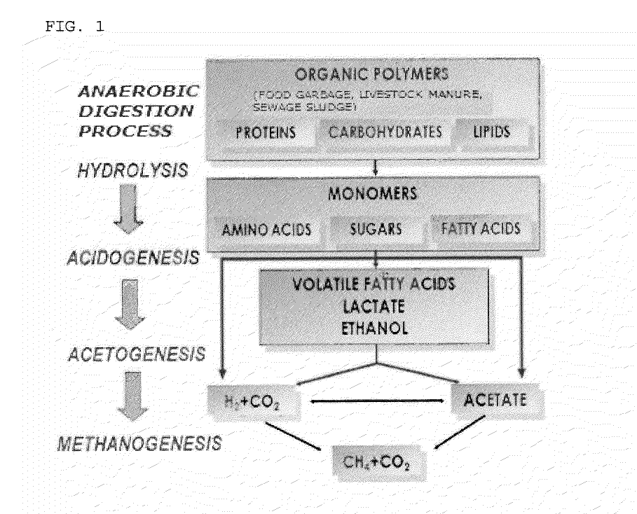

An ‘anaerobic digestion process’ refers to a process in that a biologically degradable organic compound is degraded into carbon dioxide and methane in an oxygen-free state, and anaerobic degradation to degrade and convert an organic compound to methane may typically include three separate processes, that is, hydrolysis, acidogenesis and methanogenesis stages (see FIG. 1).

The ‘hydrolysis’ stage is the first step of a fermentation process wherein organic compounds having a complicated structure (carbohydrates, proteins, lipids) are degraded by a hydrolase for hydrolysis fungus and ferment fungus, and complicated organic compounds such as polysaccharides, carbohydrates, proteins, fats, lignin, cellulose, or the like, are degraded into monomers or oligomers such as soluble sugars, alcohols, fatty acids, amino acids, polypeptides, or the like.

That is, the hydrolysis stage may be construed as a process for chain-hydrolysis of a polymer material into a low molecular weight material (i.e. monomer or oligomer) using extracellular enzymes secreted by various microorganisms, such as cellobiase, amylase, protease, lipase, etc., and is known as a rate-limiting step of overall anaerobic degradation. The hydrolysis stage is a complicated reaction affected by a shape and size of solid materials and residual concentration, in addition to pH, temperature and hydrolase. Further, the hydrolysis stage may be inhibited by the produced organic acid. In order to overcome limitations of the anaerobic reaction due to the rate-limiting step while improving efficiency of the anaerobic reaction, studies on chemical, physical or biological pre-treatment of solids have been actively conducted.

The ‘acidogenesis or fermentation’ stage is a further step in that amino acid, sugars and a part of fatty acids are further degraded to produce acetic acid, propionic acid, butyric acid and a small amount of valeric acid as well as hydrogen and carbon dioxide. Propionic acid and butyric acid are further degraded to generate a precursor for methane production, such as hydrogen, carbon dioxide and acetate. Factors affecting the present stage, that is, acidogenesis, include pH and temperature. In particular, an optimum pH range is from pH 5.7 to 6.0, and an optimum temperature is a moderate temperature of 37° C. and a high temperature of 52° C. For the moderate temperature, an acid product has a constant distribution thus being more stable at high loads, as compared to at high temperature.

The ‘methanogenesis’ stage is a process of producing methane as a final product of anaerobic degradation, which is a step of converting a substrate to methane and carbon dioxide with the help of methanogenic and non-methanogenic microorganisms. A methanogenic microorganism directly uses acetic acid, hydrogen, carbon dioxide, formic acid and methanol as a substrate, and other substrates such as fatty acids and alcohols are used by the non-methanogenic microorganism. When fat and protein are subjected to anaerobic fermentation, acetic acid is the most generated intermediate, and about 70% of methane as the final product of the fermentation is converted from acetic acid. Methanogenic microorganisms are very sensitive to environments. It is known that an optimum temperature thereof is both of a moderate temperature and a high temperature, and an optimum pH ranges from pH 7.0 to 8.0.

Such an anaerobic digestion process as described above may involve different microorganism groups until the organic compound is fermented to produce methane. Particularly, the microorganism groups may include: a hydrolytic fermentative bacteria that hydrolyzes a complicated polymer organic compound; a syntrophic acetogenic bacteria that degrades lactate, ethanol, propionate, butyrate, etc. into acetate possibly used by methanogens as well as hydrogen under a low hydrogen concentration condition; a methanogenic microorganism that ferments acetate, formate, hydrogen and carbon dioxide to produce methane; and a sulfate-reductive microorganism and denitrification bacteria that participate in a methane fermentation (‘methanogenesis’) process, and in turn, reduction of sulfur and nitrogen components.

Meanwhile, ‘pancreatin’ is a material secreted from the pancreas of an animal, mostly, pigs, which is an enzyme formulation comprising digestive enzymes such as amylase, lipase, protease, trypsin, or the like. This material is a white or pale yellow powder, has unique odor, and shows optimum activity at pH 7.0 to pH 8.0. Until the present, pancreatin is typically applied to an additive for manufacturing food, a digestive enzyme formulation, and a livestock feed additive for improving digestive efficiency, etc.

The present inventors originally established a fact that, if pancreatin having the features described above is used in an anaerobic digestion process for treatment of organic waste (i.e., livestock manure), hydrolysis efficiency is improved and, at the same time, production of biogas (methane) may be very effectively promoted.

According to the following Example 1 of the present invention, it was found that, as a result of proceeding an anaerobic digestion process by adding pancreatin to livestock manure, a gas generating time was shortened by 2-fold or more, as compared to a control group (a group without adding pancreatin).

Based on the above result, the present inventors demonstrated through experiments that pancreatin may have excellent effects of improving hydrolysis efficiency and promoting production of biogas in an anaerobic digestion process for treatment of organic waste.

In other words, as described above, a hydrolysis stage in the anaerobic digestion process is a process of hydrolyzing a polymer material into a low molecular weight material which substantially comprises a rate-limiting step of overall anaerobic degradation. It was experimentally demonstrated that pancreatin used in the present invention may effectively accelerate the progress of hydrolysis, thus rapidly shortening the anaerobic degradation.

Therefore, the present invention is characterized by providing a composition for improving hydrolysis efficiency or promoting production of biogas in an anaerobic digestion process for treatment of organic waste, which includes pancreatin as an active ingredient.

Types of the organic waste may include, for example; domestic garbage, agricultural and livestock waste, forestry waste, processed marine waste, animal/plant residues, sewage sludge, or the like. The organic waste is preferably livestock manure, and more preferably, pig manure.

The pig manure has a low content of degradation resistant materials such as cellulose, lignin, etc., and a high content of starch or protein, thus being quickly degraded, as compared to other livestock manures. However, the pig manure is typically discharged in a large amount and has a high water content of 92 to 93%, thus entailing difficulties in storage in a farm and processing the same. Since the pig manure has a nasty foul odor and high contents of BOD, COD and phosphorus, it may cause significant pollution of surrounding environments if the manure is released without alternative processing. Accordingly, there is still a need for developing technologies effectively utilized in conformance with real conditions of the country.

The above composition includes pancreatin as an active ingredient and, according to one embodiment of the present invention, the composition may have pH 7.0 to pH 8.0 at which pancreatin may express optimum activity.

Additionally, the inventive composition may further include a microorganism with excellent degradability of degradation resistant organic compounds, other than pancreatin as an active ingredient.

In general, livestock manure and food garbage contain non-structural carbohydrates such as cellulose, hemicelluloses, pectin, etc., and degradation resistant organic components such as lignin (mostly vegetable materials). Since pancreatin can catalyze only degradation of carbohydrates, proteins and fats, when a microorganism capable of degrading such degradation resistant organic compounds as described above is further added, hydrolysis efficiency may be further improved.

The microorganism with excellent ability to degrade typically degradation resistant organic compounds used in the present invention may include any of conventionally known microorganisms for degradation of degradation resistant organic compounds without particular limitation thereto, for example, bacillus subtilis EDS-1 (KCTC 18122P), novel bacillus licheniformis EDS-2 (KCTC 18128P), etc., disclosed in Korean Patent Application Laid-Open No. 2011-0019079.

The present invention also provides a method for treatment of organic waste by an anaerobic digestion process, which includes adding a composition containing pancreatin as an active ingredient, and a seed mixture; and conducting a reaction thereof.

The anaerobic digestion process of the present invention may be conducted at a low temperature (5 to 25° C.), moderate temperature (35 to 38° C.) or high temperature (55 to 60° C.) Digestion at low temperature entails problems such that a digestion speed is low and a residence time in a digestion bath ranges from 3 to 12 months, which in turn, requires a long period of time. On the other hand, for digestion at high temperature, there is a drawback of increased energy consumption for heating and/or heat retention in order to maintain the high temperature. Therefore, digestion at moderate temperature is preferably used.

Further, the anaerobic digestion process of the present invention may use a continuous system (for continuously feeding an organic compound into a digestion bath and discharging the same in an amount equal to the organic compound fed thereto) or a batch type system by continuous introduction of the organic compound.

Further, the anaerobic digestion process of the present invention may use a single-phase (a method of feeding an organic compound into a single digestion bath to conduct anaerobic digestion) or a two-phase (using separate acid fermentation tank and methane fermentation tank) in view of types of the digestion bath (reactor). However, since pancreatin used in the present invention has optimum activity at pH 7.0 to 8.0, this pH value is substantially identical to the optimum pH range of 7.0 to 8.0 for methane fermentation in the single-phase system that introduces an organic compound into a single digestion bath and conducts anaerobic digestion. Therefore, the single-phase system may be more effectively used.

The method for treatment of organic waste using an anaerobic digestion process of the present invention is characterized in that a composition containing pancreatin as an active ingredient and a seed mixture are added to the organic waste to conduct a reaction thereof.

According to one embodiment of the present invention, the organic waste may include, for example, consumer garbage, agricultural/livestock waste, forestry waste, processed marine waste, animal/plant residues, sewage sludge, or the like, preferably, livestock manure, and more preferably, pig manure.

According to another embodiment of the present invention, the composition containing pancreatin as an active ingredient may include pancreatin at a concentration of 1 to 100 μg/ml.

According to another embodiment of the present invention, the composition containing pancreatin as an active ingredient may have pH 7.0 to pH 8.0 to express optimum activity.

According to another embodiment of the present invention, the composition containing pancreatin as an active ingredient may further include a microorganism with excellent ability to degrade typically degradation resistant organic compounds, wherein the microorganism for degradation of degradation resistant organic compounds is not particularly limited in terms of species or types thereof, for example, bacillus subtilis EDS-1 (KCTC 18122P), novel bacillus licheniformis EDS-2 (KCTC 18128P), etc., disclosed in Korean Patent Application Laid-Open No. 2011-0019079 may be used.

The ‘seed mixture’ used herein refers to a mixture of different microorganisms required for the anaerobic digestion process and may include, for example, a seed agent or seed sludge.

The ‘seed agent’ means a bio-mass formed of microorganisms and culture aids for a variety of applications, which is prepared by culturing a single or combined microorganism strain and concentrating the cultured product to provide a fermentation broth, and forming the broth into a liquid or powder form. Accordingly, a microorganism group with degradability suitable for major waste components to be removed (or degraded) must be separated and reconstructed. In response to features of different constitutional components of the waste, the seed agent may also consist of multiple types of microorganism groups having a great variety of microbiological and/or biochemical properties and metabolic processes.

The ‘seed sludge’ means sludge containing seeds required for degradation in a fermentation bath in which waste degradation is performed.

The seed mixture used herein may be any one commercially available in the market, or suitably prepared by admixing different seeds depending upon types of the organic waste. In particular, since the method for treatment of organic waste according the present invention adopts an anaerobic digestion process, a seed agent useful for the anaerobic digestion process may be used. Otherwise, a seed sludge derived from an anaerobic digestion plant may also be used.

For manufacturing the seed mixture used herein, the seed mixture may be prepared by appropriately combining: a hydrolytic fermentative bacteria that hydrolyzes a complicated polymer organic compound; a syntrophic acetogenic bacteria that degrades lactate, ethanol, propionate, butyrate, etc, into acetate possibly used by methanogens as well as hydrogen under a low hydrogen concentration condition; a methanogenic microorganism that ferments acetate, formate, hydrogen and carbon dioxide to produce methane; and a sulfate-reductive microorganism and denitrification bacteria that participate in methanogenesis and, in turn, reduction of sulfur and nitrogen components.

The hydrolytic fermentative bacteria for hydrolyzing the polymer organic compound may include, for example, but not be limited to, Clostridium formicoaceticum, Clostridium thermoaceticum, Clostridium propionicum, Clostridium butryricum, Ruminococus flavefaciens, Bacteroides succinogenes, Selenomonas rumintium, or the like.

The syntrophic acetogenic bacteria may include, for example, but not be limited to, Syntrophomonas or Syntrobacter, etc.

The methanogenic microorganism may typically include four genera, i.e., Methanobacterium, Methanococcus, Methanosarcina and Methanospirillium. More particularly, Methanobacterium thermoautotrophicum, Methanobrevibacter arboriphilus, Methanococcus vanniellii, Methanospirillum hungatei, Methanosarcina barkeri, Methanosarcina mazei, Methanothrix soehngenii, Methanolobus tindarius, Methanococcoides methylutens, Methanoplanus limicola, Acetobacterium woodii, Propionibacterium arabinosum, Sarcina maxima, Butyribacterium methylotrophium, Lactobacillus amylophilus, or the like, are used without particular limitation thereto.

Hereinafter, the present invention will be described in detail by the following examples. These examples are proposed for more concretely explaining the present invention, while not limiting the scope of the present invention.

### EXAMPLE

### Example 1

### Effect of Pancreatin on Anaerobic Digestion Process

[1-1] Establishment of Hypothesis and Design Experiment

An anaerobic digestion process includes three biological/chemical digestion stages comprising hydrolysis, acidogenesis and methanogenesis. The anaerobic digestion process used in the present experiment adopted herein was an anaerobic sequencing batch reactor (ASBR) process among different processes based on configuration of a reactor and operational methods. The ASBR process is operated under a single-phase or two-phase condition based on the number of reactors. The two-phase reactor is provided with a front reactor that receives acidogens and has the purpose of acid generation, and a rear reactor that receives methanogens and has the purpose of methane generation. Hence, optimum pH and temperature for respective microorganisms may be suitably controlled. On the other hand, a single-phase reactor uses only one reactor, thus having a simple configuration and enabling easy operation. It is usually expected that the two-phase reactor has higher efficiency, however, due to difficulties in maintenance and management and experimental apparatus expenses, the single-phase condition was adopted as a processing method used for the present investigation.

Pancreatin is a material secreted from the pancreas and includes digestive enzymes such as amylase, lipase, protease, trypsin, etc. Amylase is a positive catalyst for hydrolysis of polysaccharides. Likewise, lipase, protease and trypsin are positive catalysts for hydrolysis of fats and proteins, respectively. Therefore, it was presumed that, when pancreatin is introduced into a reactor, the first stage of anaerobic digestion, that is, the progression of hydrolysis is accelerated, and it is possible to reduce time required for gas generation and completion of operation.

A. Experimental Preparations

A seed sludge, pig manure, two anaerobic reactors, an impeller, a temperature controller, heating bar, a thermistor, a syringe for sampling, a plastic container and a Tedlar bag were prepared. The pig manure was directly collected from a nearby farm. The seed sludge used herein was provided by an anaerobic digestion plant in a public livestock manure treatment facility located in Hongseong, South Chungcheong province, Korea. In particular, the seed sludge and pig manure were subjected to sampling when they were in an original state and when they were firstly fed into the reactor.

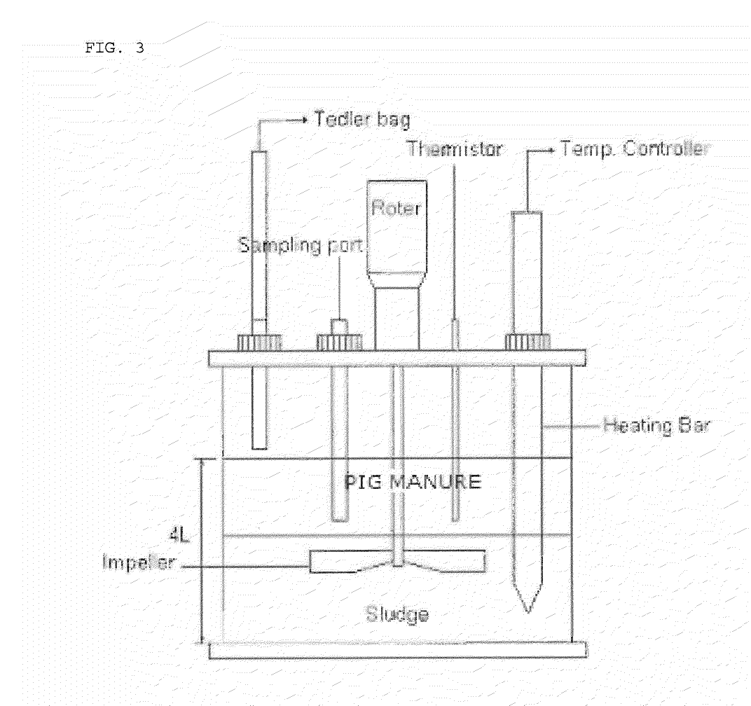

B. Design of Experiment (See FIG. 3)

(1) Experimental Condition

Effective volume: 4 L

ASBR process (batch type): pig manure (2.0 L) and seed sludge 2.0 L) were introduced in a 1:1 ratio

Estimated process operation time: considering time delay of hydrolysis stage, about 90 days

Point of time for the completion of operation: point of time at which methane is not further generated

Using a heating bar and temperature controller, temperature of a reactor is kept at 35° C.

NH4+ serving as a pH buffer is present in enough quantities in pig manure and does not need controlling.

A stirrer is controlled to rotate at 100 rpm (According to prior experiments, a motor may malfunction due to excessive loading applied thereto, which may be caused by excessively increasing voltage. Therefore, the stirrer is controlled to rotate at 80 to 90 rpm).

In order to prevent photosynthesis of sulfur bacteria, reactor is covered with aluminum foil.

For gas entrapment by water substitution, a test tube is further mounted in the middle region to prevent backflow of water.

Sampling: using a syringe, 15 mL of pig manure during igestion is extracted every 2 to 3 days

(2) Experimental Group

The same conditions as applied to a control group are used, except that pancreatin is diluted to a concentration of 10 μg/ml then introduced into the reactor (once during hydrolysis stage, then, once more again when gas was generated).

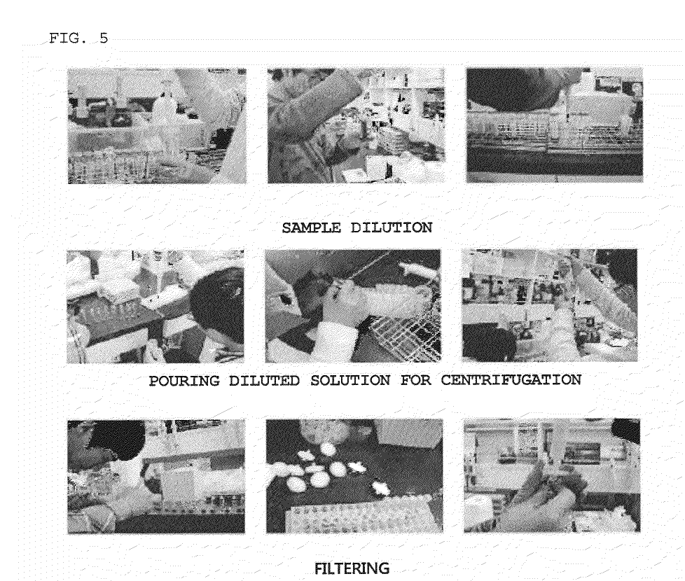

C. Measurement of Organic Acid (See FIG. 5)

(1) Alignment of sample

(2) A 1 mL sample collected using a micro-pipette is placed in a container then 9 mL of distilled water is added thereto to dilute the solution to a 10% concentration.

(3) After recording a date and details, the diluted solution is introduced in an amount of 1.5 mL per tube.

(4) After placing tubes in a centrifuge while balancing, centrifugation is performed about 20 minutes.

(5) Only supernatant of the centrifuged solution is extracted using a syringe, filtered through a filter, and collected in a labeled small vial.

(6) Assessment of the sample is requested to POSTECH Co.

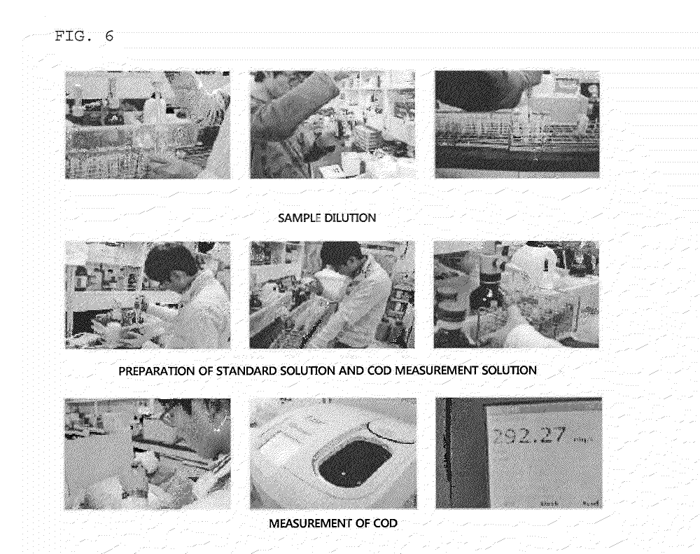

D. COD Measurement (See FIG. 6)

(1) Alignment of sample

(2) The 10% diluted solution prepared using a micro-pipette in the measurement of organic acid is further diluted to prepare a 1% diluted solution.

(3) 2.5 mL of the sample prepared in (2), 1.5 mL of COD digestion solution and 3.5 mL of sulfuric acid are placed in a vial, followed by heating the solution at 150° C. for 2 hours.

(4) Using distilled water instead of the sample, standard solutions at concentrations of 0 ppm, 250 ppm and 500 ppm, respectively, are prepared.

(5) COD content is measured after cooling the solution for 1 hour.

[1-2] Conclusion of Experiment

A. Assessment of Existing Anaerobic Digestion Process

Even at day 26 of the experiment (May 9, 2012), gas generation was not observed in the reactor according to the existing anaerobic digestion process, which was used as a control group. The above process typically includes the hydrolysis stage and acidogenesis stage for 30 to 40 days. However, even when the experiment was completed, the control group reactor was still in the hydrolysis stage or acidogenesis stage. During sampling, the sample was sometimes not easily sucked up or solids were included in the sample. The first reason for these findings is considered to be that the used pig manure was not filtered, and the second reason is considered to be that a degradation stage had not proceeded enough.

B. Effect of Pancreatin on Anaerobic Digestion Process

The reactor used as an experimental group primarily exhibited generation of bubbles at day 13 of the experiment (Apr. 26, 2012). Thereafter, bubble generation was observed almost once per day. Although the anaerobic digestion process generally needs a hydrolysis stage and acidogenesis stage for 30 to 40 days, it was found that pancreatin can noticeably reduce the duration.

Consequently, it was demonstrated that a processing time of the anaerobic digestion process using pancreatin (the experimental group) is remarkably shortened. Further, it was observed that gas was not generated in the control group even at day 26 of the experiment, while gas was continuously generated in the experimental group from day 13 of the experiment.

When both the experimental group A and the control group B were operated by the batch type and single-phase digestion processes, respectively, the experimental group having pancreatin primarily introduced therein showed a reduction in time delay in the hydrolysis stage, an increase in amount of generated methane gas, and improved removal of chemical oxygen demand (COD), From these results, it is expected that, even when processes developed since conventional digestion processes, for example, an anaerobic filter (Young and McCarty, 1969), anaerobic fluidized bed reactor (Frostell, 1982), anaerobic baffled reactor (ABR; William et al., 1999), a upflow anaerobic sludge blanket (UASB: Lettinga et al., 1980), or the like are operated in a real scale plant, effects substantially equal to those obtained by the present study may be accomplished in the same manner as described above.

As such, preferred embodiments of the present invention have been described in detail. Those skilled in the art will clearly understand various modifications of the present invention may be possibly embodied without departing from the scope and essential characteristics of the present invention. Therefore, the embodiments described herein should be considered in view of explanation rather than restrictive definition thereof. The substantial scope of the present invention is not limited to the detailed description but defined by the following claims and their equivalents, and it should be construed that all of differences within these equivalents are included in the present invention.

What is claimed is:

1. A composition for improving hydrolysis efficiency or for promoting production of biogas in an anaerobic digestion process for treatment of organic waste, comprising pancreatin as an active ingredient.

2. The composition according to claim 1, wherein the organic waste is a livestock manure.

3. The composition according to claim 1, wherein the composition has an optimum activity at pH 7.0 to pH 8.0.

4. The composition according to claim 1, further comprising a microorganism with an excellent degradability of degradation resistant organic compounds.

5. A method for treatment of organic waste using an anaerobic digestion process, comprising:
adding a composition containing pancreatin as an active ingredient and a seed mixture to the organic waste; and
conducting a reaction thereof.

6. The method according to claim 5, wherein the organic waste is a livestock manure.

7. The method according to claim 5, wherein the composition containing pancreatin as an active ingredient comprises pancreatin at a concentration of 1 to 100 μg/ml.

8. The method according to claim 5, wherein the composition containing pancreatin as an active ingredient has effects of improving hydrolysis efficiency or promoting production of biogas.

9. The method according to claim 5, wherein the composition containing pancreatin as an active ingredient has an optimum activity at pH 7.0 to pH 8.0.

10. The method according to claim 5, wherein the composition containing pancreatin as an active ingredient further comprises a microorganism with an excellent degradability of degradation resistant organic compounds.
